(19) United States
(12) Patent Application Publication
(10) Pub. No.: US 2015/0073345 A1
(43) Pub. Date: Mar. 12, 2015

(54) MEDICAL INSTRUMENT

(71) Applicant: SEIKO EPSON CORPORATION, Tokyo (JP)

(72) Inventors: Takahiro MATSUZAKI, Shiojiri-shi (JP); Kunio TABATA, Shiojiri-shi (JP); Shinichi MIYAZAKI, Suwa-shi (JP); Takeshi SETO, Chofu-shi (JP)

(21) Appl. No.: 14/483,585

(22) Filed: Sep. 11, 2014

(30) Foreign Application Priority Data
Sep. 11, 2013 (JP) 2013-188138

(51) Int. Cl. A61M 5/36; A61M 5/145

(52) U.S. Cl. CPC A61M 5/365; A61M 5/145; A61M 2205/0294
USPC 604/123; 604/131

(57) ABSTRACT

A medical instrument for ejecting liquid includes a liquid chamber that contains the liquid, a driving unit that pressurizes the liquid contained in the liquid chamber, and a vibration detector that detects vibration when the driving unit is driven.

This application claims the benefit of Japanese Patent Application No. 2013-188138, filed on Sep. 11, 2013. The content of the aforementioned patent application is incorporated by reference herein in its entirety.

### BACKGROUND

1. Technical Field

The present invention relates to a technique for a medical instrument for ejecting liquid.

2. Related Art

A medical instrument that ejects liquid from the injection tube by pressurizing the liquid with a driving unit is known. In such a medical instrument, for example, if bubbles are present in the driving unit, the pressure applied to the liquid by the driving unit is absorbed by the change in the volume of bubbles. Accordingly, there is a problem in that the liquid is not pressurized appropriately. As a technique of detecting bubbles in the liquid, the technique disclosed in JP-A-05-305141 is known.

In connection with such a medical instrument, it is an issue to develop a technique of detecting various states of the equipment regardless of the presence of bubbles in the driving unit.

In addition, when applying the technique disclosed in JP-A-05-305141 to a liquid ejection device, a sensor that can generate an ultrasonic wave and a sensor that can receive an ultrasonic wave are required. This causes a problem in that the structure is complicated and the cost is high.

### SUMMARY

An advantage of some aspects of the invention is to solve at least a part of the problems described above, and the invention can be implemented as the following aspects.

(1) An aspect of the invention provides a medical instrument for ejecting liquid. The medical instrument includes: a liquid chamber that contains the liquid; a driving unit that pressurizes the liquid contained in the liquid chamber; and a vibration detector that detects vibration when the driving unit is driven. According to the medical instrument of this aspect, it is possible to detect the vibration when the driving unit vibrates using the vibration detector. Therefore, for example, it is possible to detect the state of the medical instrument using the detected vibration.

(2) The medical instrument described above may further include a bubble detector that detects bubbles in the liquid chamber based on the vibration detected by the vibration detector. According to the medical instrument of this aspect, since the bubbles in the liquid chamber are detected based on the vibration detected by the vibration detector, it is possible to detect bubbles with a relatively simple structure. Here, the bubbles include not only spherical gas but also gas that is present separated from the liquid.

(3) In the medical instrument described above, the vibration detector may be a sound collection device. According to the medical instrument of this aspect, since the vibration detector is a sound collection device, a simple configuration is possible.

(4) In the medical instrument described above, the vibration detector may be a vibration sensor. According to the medical instrument of this aspect, since the vibration detector is a vibration sensor, a simple configuration is possible.

(5) In the medical instrument described above, the driving unit may be a piezoelectric element, and the piezoelectric element may function as the vibration detector. According to the medical instrument of this aspect, since the same piezoelectric element can function as the driving unit and the vibration detector, it is possible to simplify the structure.

(6) The medical instrument described above may further include a degassing unit that discharges bubbles in the liquid chamber to outside based on a detection result of the bubble detector. According to the medical instrument of this aspect, since the bubbles present in the liquid chamber can be discharged by the degassing unit, the driving unit can pressurize the liquid appropriately.

All of the components provided in the medical instrument described above are not essential, and some of the components may be appropriately changed, removed, or replaced with other new components and some of the limitative content may be appropriately deleted in order to solve some or all of the problems described above or to achieve some or all of the effects described in this specification. In addition, in order to solve some or all of the problems described above or to achieve some or all of the effects described in this specification, some or all of the technical features included in the aspect of the invention described above may be combined with some or all of the technical features included in the other aspects of the invention described above to realize an independent aspect of the invention.

For example, an aspect of the invention can be implemented as a device including one or more of three elements of the liquid chamber, the driving unit, and the vibration detector. That is, this device may include the liquid chamber, or may not include the liquid chamber. In addition, this device may include the driving unit, or may not include the driving unit. In addition, this device may include the vibration detector, or may not include the vibration detector. The liquid chamber may be configured as a liquid chamber that contains the liquid. The driving unit may be configured as a driving unit that pressurizes the liquid contained in the liquid chamber. The vibration detector may be configured as a vibration detector that detects vibration when the driving unit is driven. For example, such a device can be implemented not only as a medical instrument but also as devices other than the medical instrument. According to such a form, it is possible to solve at least one of the various problems relevant to the miniaturization of a device, low cost, resource saving, ease of manufacture, improvement in usability, and the like. Some or all of the technical features of each aspect of the medical instrument described above can be applied to this device.

The invention can also be implemented as various forms other than the device. For example, the invention can be implemented as forms, such as a liquid ejection device, a method of ejecting liquid, a method of manufacturing a liquid ejection device, a bubble detection method, and a bubble discharge method.

### BRIEF DESCRIPTION OF THE DRAWINGS

The invention will be described with reference to the accompanying drawings, wherein like numbers reference like elements.

FIG. 1 is an explanatory diagram illustrating the configuration of a medical instrument.

FIG. 2 is a block diagram showing the configuration of a bubble detector.

FIG. 3 is a timing chart showing the operation of the bubble detector.

FIGS. 4A and 4B are explanatory diagrams showing the measurement result of a vibration signal.

FIG. 5 is a flowchart showing the flow of a degassing process.

FIG. 6 is an explanatory diagram showing the configuration of a medical instrument of a second embodiment.

FIG. 7 is an explanatory diagram showing the configuration of a medical instrument of a third embodiment.

### DESCRIPTION OF EXEMPLARY EMBODIMENTS

### A. First Embodiment

### A1. Configuration of Medical Instrument

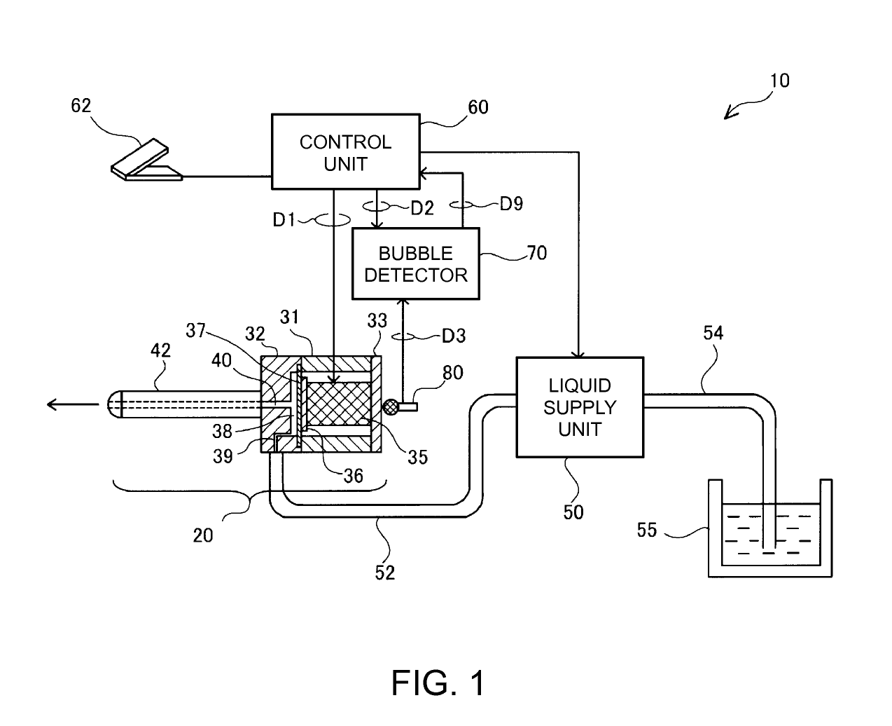

FIG. 1 is an explanatory diagram illustrating the configuration of a medical instrument 10 according to a first embodiment of the invention. The medical instrument 10 is used as a scalpel for incising or resecting the affected part by ejecting liquid to the affected part.

The medical instrument 10 includes a liquid ejection device 20, a liquid supply unit 50, a liquid container 55, a control unit 60, a bubble detector 70, and a sound collection device 80. The liquid ejection device 20 and the liquid supply unit 50 are connected to each other through a liquid supply passage 52. The liquid supply unit 50 and the liquid container 55 are connected to each other through a connection tube 54. In the present embodiment, the liquid supply passage 52 and the connection tube 54 are formed of resin.

The liquid container 55 contains a physiological saline solution as a liquid. As a liquid, it is possible to use various liquids, such as sterile water for medical use or pure water. The liquid supply unit 50 supplies liquid, which is sucked from the liquid container 55 through the connection tube 54, to the liquid ejection device 20 through the liquid supply passage 52.

The liquid ejection device 20 applies pulsation to the liquid supplied from the liquid supply unit 50, thereby ejecting pulsed liquid. The user incises or resects the affected part by applying the pulsed liquid ejected from the liquid ejection device 20 to the affected part of the patient.

The liquid ejection device 20 includes a first case 31, a second case 32, a third case 33, a piezoelectric element 35, a reinforcing plate 36, a diaphragm 37, and an injection tube 42. The first case 31 is a cylindrical member. One end of the first case 31 is bonded to the second case 32. The other end of the first case 31 is closed by the third case 33. The piezoelectric element 35 is disposed in a space formed inside the first case 31.

The piezoelectric element 35 is a laminated piezoelectric element. One end of the piezoelectric element 35 is fixed to the diaphragm 37 through the reinforcing plate 36. The other end of the piezoelectric element 35 is fixed to the third case 33. The diaphragm 37 is formed of a metal thin film, and a peripheral portion of the diaphragm 37 is fixed to the first case 31. A liquid chamber 38 is formed between the diaphragm 37 and the second case 32. The volume of the liquid chamber 38 is changed by the driving of the piezoelectric element 35.

A first flow passage 39 for making liquid flow into the liquid chamber 38 is formed in the second case 32. The first flow passage 39 is connected to the liquid supply passage 52. The liquid supplied from the liquid supply unit 50 flows into the liquid chamber 38 through the liquid supply passage 52 and the first flow passage 39. In addition, a second flow passage 40 for discharging the liquid contained in the liquid chamber 38 is formed in the second case 32. The second flow passage 40 is connected to the injection tube 42.

The control unit 60 controls the overall operation of the medical instrument 10. A foot switch 62 operated by the user with a foot is connected to the control unit 60. When the user turns on the foot switch 62, the control unit 60 controls the liquid supply unit 50 to supply the liquid to the liquid ejection device 20 (liquid chamber 38), and transmits a driving signal to the piezoelectric element 35. When the driving signal is received from the control unit 60, the piezoelectric element 35 vibrates at a predetermined frequency. When the piezoelectric element 35 vibrates, the volume of the liquid chamber 38 is changed through the diaphragm 37, and the liquid contained in the liquid chamber 38 is pressurized. Pulsation is applied to the liquid pressurized at a predetermined frequency, and the liquid is ejected outside as a pulsed liquid through the second flow passage 40 and the injection tube 42.

The ejection of pulsed liquid means the ejection of liquid in a state where the flow rate or the flow velocity changes. The ejection of pulsed liquid includes intermittent ejection in which the liquid is ejected while repeating ejection and stopping. However, since it is sufficient that the flow rate or the flow velocity of the liquid is changed, the intermittent ejection does not necessarily need to be adopted.

The sound collection device 80 as a vibration detector collects vibration (in the present embodiment, acoustic vibration) generated from the liquid ejection device 20 by the driving of the piezoelectric element 35. The sound collection device 80 converts the collected vibration into an electrical signal, and inputs the electrical signal to the bubble detector 70 as a vibration signal D3. The bubble detector 70 detects the presence of bubbles or the amount of bubbles in the liquid chamber 38 based on the vibration signal D3. When the bubble detector 70 detects bubbles in the liquid chamber 38, the control unit 60 performs a degassing process for discharging the bubbles in the liquid chamber 38 to the outside. The degassing process will be described later.

### A2. Configuration of a Bubble Detector

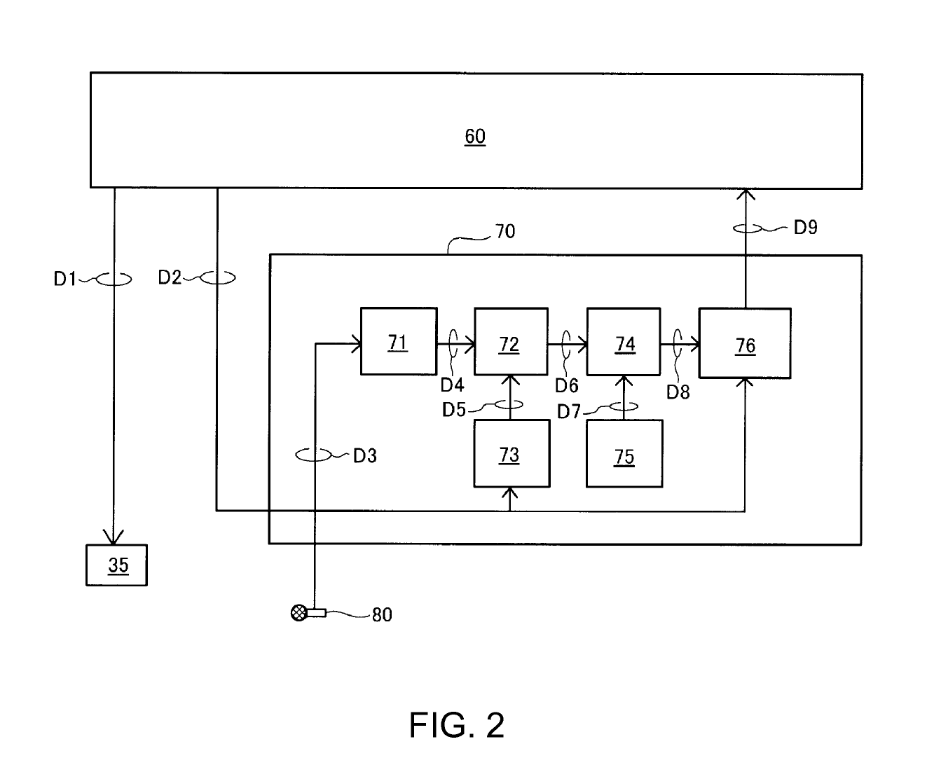

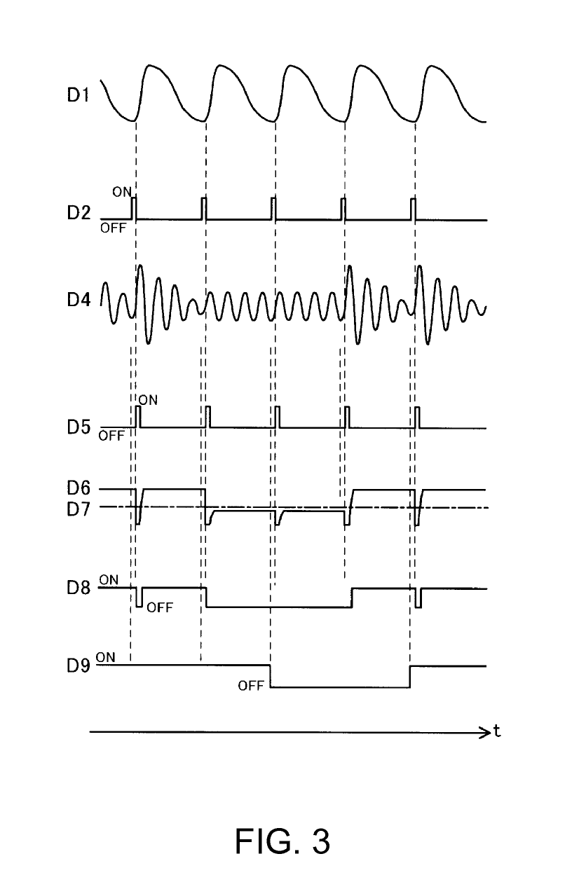

The details of the configuration and operation of the bubble detector 70 will be described with reference to FIGS. 2 and 3. FIG. 2 is a block diagram showing the configuration of the bubble detector 70. FIG. 3 is a timing chart showing the operation of each component provided in the bubble detector 70.

As shown in FIG. 2, the bubble detector 70 includes a band pass filter 71, a peak hold circuit 72, a delay circuit 73, a comparator 74, a reference voltage generator 75, and a latch 76. A timing signal D2 and the vibration signal D3 are input to the bubble detector 70. The timing signal D2 is input to the bubble detector 70 from the control unit 60. The timing signal D2 is a binary signal synchronized with a driving signal D1 that is input from the control unit 60 to the piezoelectric element 35 (refer to FIG. 3). The vibration signal D3 is input to the bubble detector 70 from the sound collection device 80. The vibration signal D3 is a signal obtained when the sound collection device 80 converts the sound generated from the liquid ejection device 20 into an electrical signal.

The band pass filter 71 receives the vibration signal D3, extracts only a signal of a predetermined frequency band, and inputs the signal to the peak hold circuit 72 as a specific frequency signal D4. The peak hold circuit 72 stores the peak value of the specific frequency signal D4 input from the band pass filter 71. The peak hold circuit 72 inputs a peak value signal D6, which shows the stored peak value of the specific frequency signal D4 as a voltage value, to the comparator 74.

The comparator 74 compares the peak value signal D6 input from the peak hold circuit 72 with a predetermined reference voltage value. The reference voltage value is generated by the reference voltage generator 75, and is input to the comparator 74 as a reference voltage signal D7. The comparator 74 compares the voltage value of the peak value signal D6 with the voltage value of the reference voltage signal D7, and outputs the comparison result to the latch 76 as a binary signal (hereinafter, referred to as a comparison signal D8). The comparator 74 sets the value of the comparison signal D8 to ON when the voltage value of the peak value signal D6 is higher than the reference signal.

The comparison signal D8 and the timing signal D2 are input to the latch 76. The latch 76 reads the value of the comparison signal D8 at the timing when the timing signal D2 is ON, and inputs the read value to the control unit 60 as a bubble detection signal D9.

The delay circuit 73 receives the timing signal D2 from the control unit 60. The delay circuit 73 inputs a signal obtained by delaying the timing signal D2 (hereinafter, also referred to as a clear signal D5) to the peak hold circuit 72. The peak hold circuit 72 clears the stored peak value of the specific frequency signal D4 in synchronization with the clear signal D5.

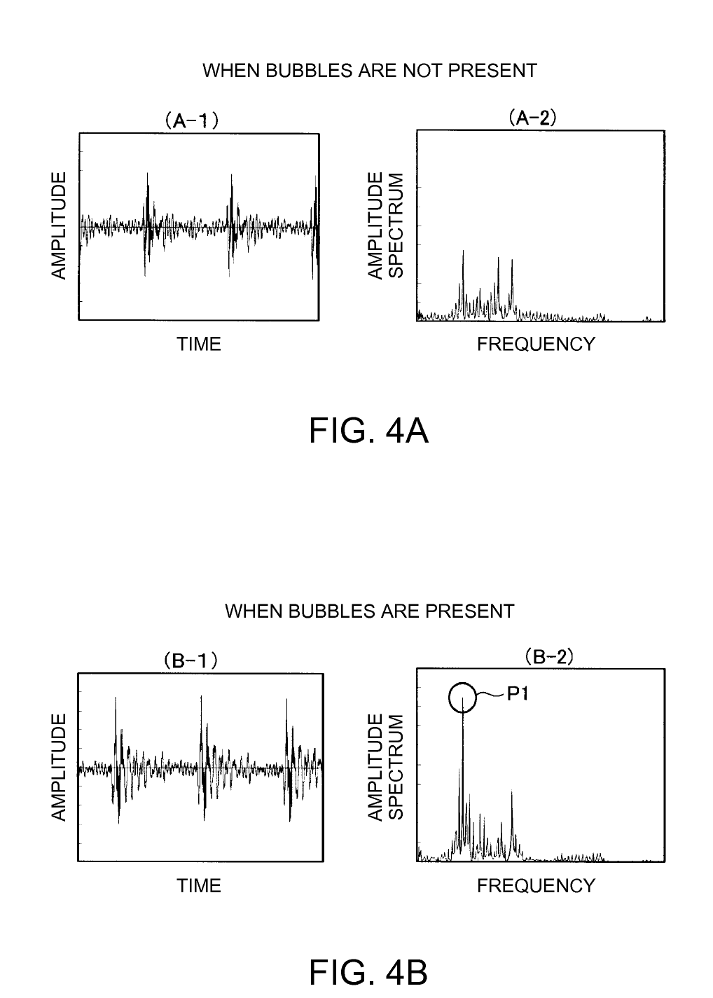

Here, the vibration signal D3 will be described. As described above, the vibration signal D3 is a signal obtained when vibration (in the present embodiment, acoustic vibration) generated from the liquid ejection device 20 by the driving of the piezoelectric element 35 is collected and is converted into an electrical signal by the sound collection device 80. FIGS. 4A and 4B are explanatory diagrams showing the measurement result of the actual vibration signal D3. FIG. 4A shows the vibration signal D3 when there are no bubbles in the liquid chamber 38. FIG. 4B shows the vibration signal D3 when bubbles are present in the liquid chamber 38. (A-1) and (B-1) in FIGS. 4A and 4B show the vibration signal D3 where the horizontal axis indicates time and the vertical axis indicates amplitude. (A-2) and (B-2) in FIGS. 4A and 4B show the vibration signal D3 where the horizontal axis indicates frequency and the vertical axis indicates amplitude spectrum (sound pressure spectrum).

As can be seen from the comparison between (A-1) and (B-1) in FIGS. 4A and 4B, the peak value of the amplitude of the vibration signal D3 when bubbles are present in the liquid chamber 38 is larger than that when bubbles are not present in the liquid chamber 38. In addition, as can be seen from the comparison between (A-2) and (B-2) in FIGS. 4A and 4B, a large peak (peak P1 in the diagram) is observed in the amplitude spectrum (sound pressure spectrum) of a specific frequency component. In this measurement, the frequency of the peak P1 was 3.7 kHz. Thus, the characteristics of the vibration generated by the liquid ejection device 20 differ depending on the presence of bubbles or the amount of bubbles in the liquid chamber 38.

The bubble detector 70 detects the presence of bubbles or the amount of bubbles in the liquid chamber 38 by detecting the peak P1. Specifically, the bubble detector 70 can detect the presence of bubbles or the amount of bubbles in the liquid chamber 38 by setting the pass band of the band pass filter 71 of the bubble detector 70 to a frequency band including the frequency of the peak P1 and setting the reference voltage input to the comparator 74 to a value by which the peak P1 can be detected.

### A3. Degassing Process

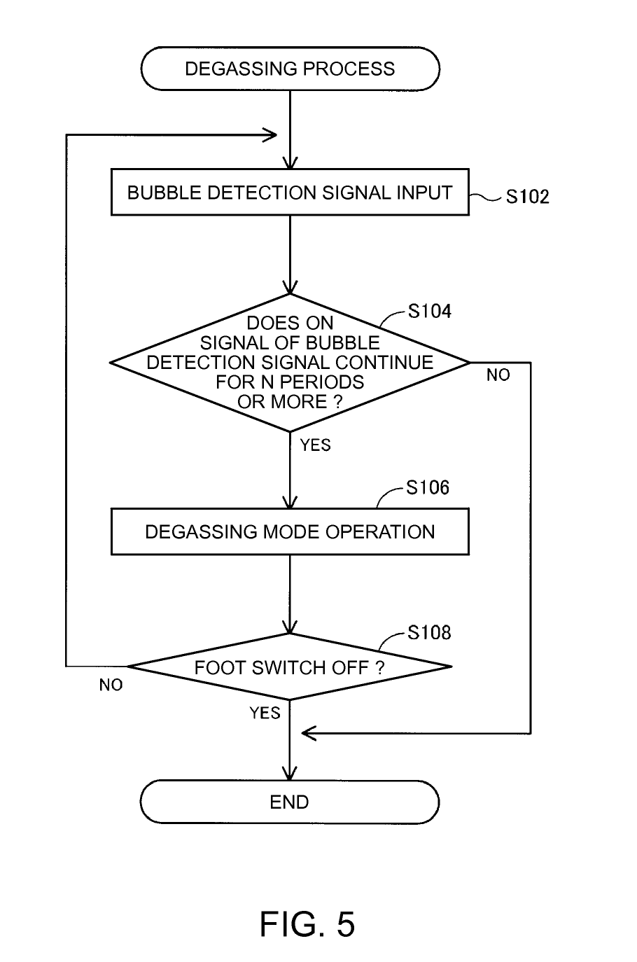

A degassing process performed by the control unit 60 will be described. The degassing process is a process for discharging bubbles present in the liquid chamber 38 to the outside. FIG. 5 is a flow chart showing the flow of the degassing process. The degassing process starts when the user of the medical instrument 10 turns ON the foot switch 62. When the degassing process starts, the control unit 60 operates the bubble detector 70 and receives the bubble detection signal D9 from the bubble detector 70 (step S102). The control unit 60 reads the value of the bubble detection signal D9 and determines whether or not the ON signal of the bubble detection signal D9 continues for N periods (step S104). As the period, a period of the vibration signal D3 is used. N is a value set in the control unit 60 in advance. In the present embodiment, N is determined by measuring the relationship between the presence of bubbles or the amount of bubbles in the liquid chamber 38 and the vibration signal D3.

When the control unit 60 determines that the ON signal of the bubble detection signal D9 continues for N periods (step S104: YES), the control unit 60 performs a degassing mode operation for a predetermined time (step S106). In the present embodiment, as a degassing mode operation, the control unit 60 controls the liquid supply unit 50 to increase the flow rate of the liquid, which is supplied to the liquid ejection device 20, from the flow rate of the liquid at the time of normal operation. In addition, the control unit 60 changes the voltage value and the frequency of the driving signal D1. As the flow rate of the liquid supplied to the liquid ejection device 20 and the voltage value of the driving signal D1 applied to the piezoelectric element 35, values increased within the range where the safe operation is possible are used. Due to the degassing mode operation performed by the control unit 60, bubbles in the liquid chamber 38 are discharged to the outside. The control unit 60 performs the process of steps S102 to S106 repeatedly until the user turns OFF the foot pedal (step S108).

As described above, the medical instrument 10 detects the presence of bubbles or the amount of bubbles in the liquid chamber 38 based on the vibration (in the present embodiment, acoustic vibration) generated by the driving of the piezoelectric element 35. Therefore, it is possible to detect the presence of bubbles or the amount of bubbles in the liquid chamber 38 with a relatively simple configuration, such as the sound collection device 80 and the bubble detector 70.

In the medical instrument 10, when bubbles are detected, a degassing mode operation is performed to discharge the bubbles from the liquid chamber 38. Accordingly, the piezoelectric element 35 can pressurize the liquid of the liquid chamber 38 appropriately. Since the bubble detector 70 can be formed by an electrical circuit, it is possible to detect bubbles with a simple configuration and at low cost.

### B. Second Embodiment

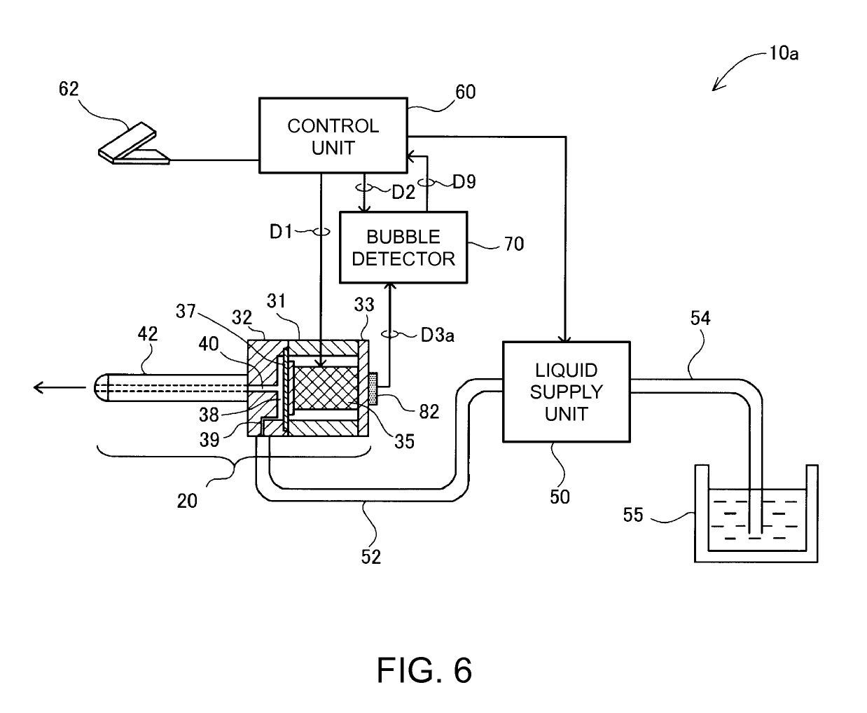

A second embodiment of the invention will be described. FIG. 6 is an explanatory diagram showing the configuration of a medical instrument 10a of the second embodiment. The second embodiment is different from the first embodiment in that a vibration sensor 82 is adopted as a vibration detector. Since the other components of the medical instrument 10a are the same as those of the medical instrument 10 in the first embodiment, the configuration of the medical instrument 10a other than the vibration sensor 82 is omitted.

The vibration sensor 82 is fixed to the third case 33, and detects the vibration of the liquid ejection device 20. The vibration sensor 82 converts the detected vibration into an electrical signal, and inputs the electrical signal to the bubble detector 70 as a vibration signal D3a. In the present embodiment, the vibration sensor 82 is a piezoelectric element. The vibration signal D3a generated by the vibration sensor 82 is the same as the vibration signal D3 generated by the sound collection device 80 in the first embodiment, and the vibration characteristics differ depending on the presence of bubbles or the amount of bubbles in the liquid chamber 38.

For example, when bubbles are not present in the liquid chamber 38, a reaction force is received from the liquid in the liquid chamber when the piezoelectric element 35 pressurizes the liquid chamber 38. Due to the reaction force, strain occurs in the third case 33, and this appears as a vibration waveform. On the contrary, when bubbles are present in the liquid chamber 38, the pressure applied to the liquid by the piezoelectric element 35 is absorbed by the change in the volume of bubbles, and the reaction force that the piezoelectric element 35 receives from the liquid is reduced. Accordingly, the strain of the third case 33 is also reduced to change the vibration characteristics. The amount of reduction of the reaction force that the piezoelectric element 35 receives from the liquid changes with the amount of bubbles. In this case, the amount of strain of the third case 33 is also different.

In the present embodiment, as in the first embodiment, the vibration characteristics appearing in the vibration signal D3a when bubbles are present in the liquid chamber 38 are detected by the bubble detector 70. Specifically, the bubble detector 70 can detect the vibration characteristics of the vibration signal D3a when bubbles are present in the liquid chamber 38 by adjusting the pass band of the band pass filter 71 and the voltage value of the reference voltage input to the comparator 74 from the reference voltage generator 75. The control unit 60 performs a degassing process based on the bubble detection signal D9 input from the bubble detector 70.

As described above, the medical instrument 10a in the second embodiment detects the vibration generated by the driving of the piezoelectric element 35 using the vibration sensor 82. Therefore, a vibration detector having a relatively simple structure is possible. By adopting the small vibration sensor 82, it is possible to miniaturize the liquid ejection device 20.

### C. Third Embodiment

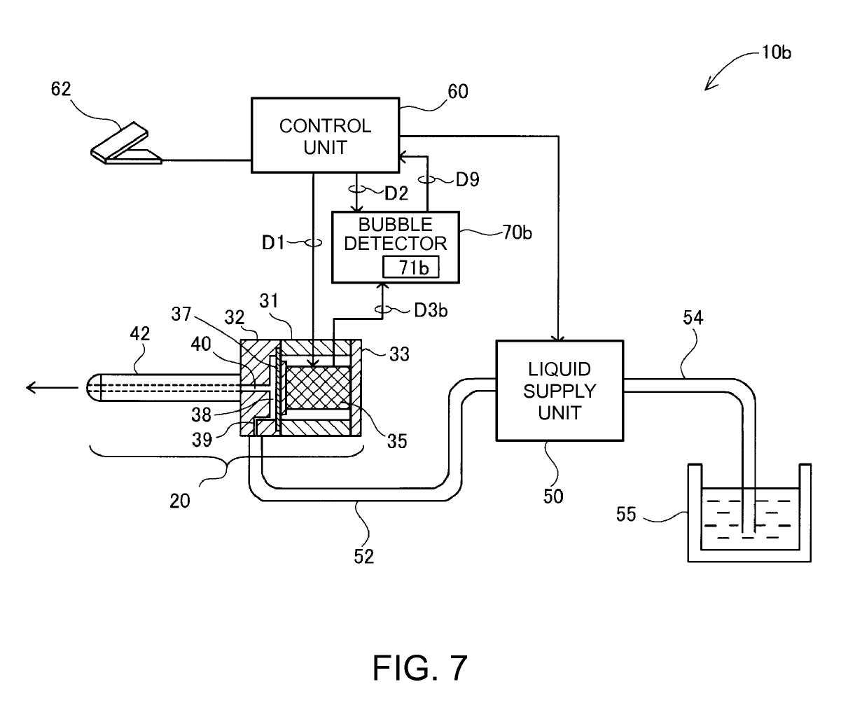

A third embodiment of the invention will be described. FIG. 7 is an explanatory diagram showing the configuration of a medical instrument 10b of the third embodiment. The third embodiment is different from first embodiment in that the piezoelectric element 35 is adopted as a vibration detector. That is, the piezoelectric element 35 has a function as a driving unit and a function as a vibration detector.

As shown in FIG. 7, a driving signal D1 is input to the piezoelectric element 35 from the control unit 60. The piezoelectric element 35 inputs a vibration signal D3b to a bubble detector 70b. The vibration signal D3b is equivalent to the back electromotive force of the piezoelectric element 35. A frequency component due to the vibration of the liquid ejection device 20 detected by the piezoelectric element 35 and a frequency component corresponding to the driving signal D1 are included in the vibration signal D3b. The bubble detector 70b includes a band pass filter 71b, and removes the frequency component corresponding to the driving signal D1 from the vibration signal D3b by adjusting the pass band and extracts the vibration characteristics appearing in the vibration signal D3b when bubbles are present in the liquid chamber 38. Although the removal of the frequency component corresponding to the driving signal D1 and the extraction of the vibration characteristics when bubbles are present in the liquid chamber are performed by one band pass filter in the present embodiment, the removal and the extraction may be performed by separate band pass filters.

By detecting the vibration characteristics extracted by the band pass filter 71b, the bubble detector 70b can detect bubbles in the liquid chamber 38. The bubble detector 70b inputs a detection result of the bubbles in the liquid chamber 38, as the bubble detection signal D9, to the control unit 60. The control unit 60 determines the presence of bubbles or the amount of bubbles in the liquid chamber 38 based on the bubble detection signal D9, and performs a degassing process.

As described above, in the medical instrument 10b of the third embodiment, the piezoelectric element 35 has a function as a driving unit and a function as a vibration detector. Therefore, it is possible to miniaturize and simplify the structure of the medical instrument 10b. In addition, since it is not necessary to prepare a vibration detector separately, it is possible to realize a low cost.

### D. Modification Examples

In addition, the invention is not limited to the above-described embodiments, but various modifications can be made within the scope without departing from the subject matter or spirit of the invention. For example, the following modification examples are also possible.

### D1. Modification Example 1

In the embodiments described above, bubbles in the liquid chamber 38 are detected based on the detected vibration of the liquid ejection device 20. However, various states of the liquid ejection device 20 can be detected based on the detected vibration of the liquid ejection device 20. For example, when cracking occurs in a part of the liquid ejection device 20 (for example, the diaphragm 37 or the first case 31) or when a water leak occurs due to cracking, the waveform of the detected vibration of the liquid ejection device 20 is different from that in the normal state. For example, when the state of connection between the first flow passage 39 and the liquid supply passage 52 is different from that in the normal state, the waveform of the detected vibration of the liquid ejection device 20 is different from that in the normal state. For example, when a bolt (screw) used in the liquid ejection device 20 is loose, the waveform of the detected vibration of the liquid ejection device 20 is different from that in the normal state. Thus, various states of the liquid ejection device 20 can be detected by comparing the detected vibration of the liquid ejection device 20 with the vibration in the normal state or by analyzing the vibration waveform. The medical instrument 10 may include a state detector that detects various states of the liquid ejection device 20. The state detector has a function of detecting various states of the liquid ejection device 20 in addition to the function of the bubble detector.

When the state detector has detected a change in the state of the liquid ejection device 20 based on the vibration of the liquid ejection device 20, it is also possible to perform control to stop the operation of the liquid ejection device 20.

### D2. Modification Example 2

In the embodiments described above, a piezoelectric element is adopted as a vibration sensor. However, it is possible to adopt various vibration sensors that detect the vibration of the liquid ejection device 20, such as an electrostrictive element or a vibration sensor that emits laser light to the liquid ejection device 20 and detects the vibration of the liquid ejection device 20 from the behavior of reflected light.

### D3. Modification Example 3

In the embodiments described above, as a degassing method, (1) increase in the flow rate of the liquid supplied to the liquid ejection device 20, (2) increase in the voltage value of the driving signal D1, and (3) change of the frequency of the driving signal D1 are performed. As a degassing method, one or two of the three methods may be adopted, or two or more methods may be combined. Also in these cases, it is possible to discharge bubbles in the liquid chamber 38.

### D4. Modification Example 4

In the embodiments described above, a piezoelectric element is adopted as a driving unit. However, it is possible to adopt various driving units capable of pressurizing the liquid contained in the liquid chamber, such as an electrostrictive element or a driving motor.

### D5. Modification Example 5

In the embodiments described above, the vibration detector is adopted. However, instead of providing the vibration detector, a degassing instruction may be input to the control unit 60 so that (1) increase in the flow rate of the liquid supplied to the liquid ejection device 20, (2) increase in the voltage value of the driving signal D1, and (3) change of the frequency of the driving signal D1 are performed for a predetermined time as a degassing mode operation. As a degassing method, one or two of the three methods may be adopted, or two or more methods may be combined. Also in these cases, it is possible to discharge bubbles in the liquid chamber 38.

Although the liquid ejection device has been described in the above embodiments, the invention is not limited to the liquid ejection device. For example, the invention can also be applied to a liquid circulation device from which liquid is discharged and to which the liquid flows. In addition, in the case of a container that contains liquid, the presence of bubbles or the amount of bubbles can be checked by applying vibration to the liquid directly or indirectly and detecting the vibration. Therefore, this is suitable for a medical instrument for which high reliability or safety is required.

What is claimed is:

1. A medical instrument for ejecting liquid, comprising:
a liquid chamber that contains the liquid;
a driving unit that pressurizes the liquid contained in the liquid chamber; and
a vibration detector that detects vibration when the driving unit is driven.

2. The medical instrument according to claim 1, further comprising:
a bubble detector that detects bubbles in the liquid chamber based on the vibration detected by the vibration detector.

3. The medical instrument according to claim 1,
wherein the vibration detector is a sound collection device.

4. The medical instrument according to claim 1,
wherein the vibration detector is a vibration sensor.

5. The medical instrument according to claim 1,
wherein the driving unit is a piezoelectric element, and
the piezoelectric element functions as the vibration detector.

6. The medical instrument according to claim 2, further comprising:
a degassing unit that discharges bubbles in the liquid chamber to outside based on a detection result of the bubble detector.
